(19) United States
(12) Patent Application Publication
(10) Pub. No.: US 2022/0347334 A1
(43) Pub. Date: Nov. 3, 2022

(54) STERILIZATION AND DISINFECTION DEVICE AGAINST BACTERIA AND VIRUSES

(71) Applicant: Yantai United Ozonetec Corporation, Yantai (CN)

(72) Inventors: Ming-Yung Hsu, Yantai (CN); Wei-Chun Hsu, Yantai (CN)

(21) Appl. No.: 17/243,422

(22) Filed: Apr. 28, 2021

(51) Int. Cl. A61L 9/12; F24F 6/06

(52) U.S. Cl. CPC A61L 9/122; F24F 6/06; A61L 2209/13; A61L 2209/212; F24F 2006/008

(57) ABSTRACT

A sterilization and disinfection device includes a housing. A control board, a humidification water tank, a humidification water trough, an ultrasonic humidifier and an ozone water tank are provided inside the housing. The humidification water trough communicates with a humidification outlet through a humidification pipe. A humidification air input channel is connected to a humidification fan connected to a humidification air inlet. The ozone water tank is equipped with an electrolytic ozone generator. The ozone water tank communicates with an ozone gas outlet through an ozone gas output pipe and a mixing chamber. The ozone water tank communicates with a water filling port through a water filling pipe. An ozone fan is installed in an ozone air inlet. The control board is electrically connected to the button display panel, the ultrasonic humidifier, the electrolytic ozone generator, the ozone fan, the humidification fan and the power inlet, respectively.

### FIELD OF THE INVENTION

The present invention relates to a sterilization and disinfection device against bacteria and viruses, and more particularly an ozone sterilization and disinfection device.

### BACKGROUND OF THE INVENTION

The indoor spaces of hotel rooms and home rooms are enclosure spaces, with poor air circulation. Current events have revealed to the general public that with the accumulation of viruses and bacteria it is easy to cross-infect and accelerate the infection. This is especially relevant under the current serious situation of the new novel coronavirus. A research group led by Professor Takayuki Murata (Virus and Parasitology) of Fujita Medical University has found that even low concentrations (0.05 or 0.1 ppm) of ozone gas can be used against the novel coronavirus. This study confirmed that under high moisture conditions there is an improved decontamination effect when using ozone gas. In a room with high moisture, even if there is a person, it is possible to reduce the spread of the bacteria and viruses by continuously implementing low-concentration ozone gas treatment.

In view of this, the inventor of the present invention has devoted himself based on his many years of practical experiences to find a solution that can reduce the spread of the new novel coronavirus and other such bacteria an viruses to reduce the possibility of infection.

### SUMMARY OF THE INVENTION

In order to help alleviate the current pandemic and help reduce the spread of harmful bacteria and viruses the present invention is to overcome the above-mentioned shortcomings of the prior art and provide a sterilization and disinfection device against the new novel coronavirus and other harmful virus and bacteria with humidification and ozone sterilization functions.

A sterilization and disinfection device comprises a housing. The housing has a power inlet and a button display panel thereon. A control board is provided inside the housing. A humidification water tank is provided inside the housing. The humidification water tank is arranged above a humidification water trough. The humidification water tank has a mouth to be inserted into the humidification water trough. An ultrasonic humidifier is mounted to a bottom of the humidification water trough. One end of a humidification pipe is connected to an upper outlet of the humidification water trough, and another end of the humidification pipe is connected to a humidification outlet on the housing. One end of a humidification air input channel is connected to an upper end of the humidification water trough, and another end of the humidification air input channel is connected to a humidification fan. The humidification fan is installed in a humidification air inlet on the housing. An ozone water tank is provided inside the housing. An electrolytic ozone generator is mounted to a bottom of the ozone water tank. One end of an ozone gas output pipe is connected to a top of the ozone water tank, and another end of the ozone gas output pipe is connected to a mixing chamber. A top of the mixing chamber communicates with an ozone gas outlet. One end of a water filling pipe is connected to the top of the ozone water tank, and another end of the water filling pipe is connected to a water filling port on the housing. An ozone fan is mounted to one side of the mixing chamber. The ozone fan is located in an ozone air inlet on the housing. The control board is electrically connected to the button display panel, the ultrasonic humidifier, the electrolytic ozone generator, the ozone fan, the humidification fan and the power inlet, respectively.

Preferably, when there is a person in an indoor space, the current of the sterilization and disinfection device is controlled to be decreased so as to decrease ozone gas to be generated for an ozone concentration to be less than 0.08 ppm. When there is no person in an indoor space, the current of the sterilization and disinfection device is controlled to be increased so as to increase ozone gas to be generated for an ozone concentration to be 0.1-0.5 ppm.

The beneficial effect of the present invention is that the present invention generates ozone gas by electrolyzing pure water through the ozone generator. Ozone gas passes through the ozone gas output pipe to the ozone gas outlet. The ozone fan sends the air to the ozone gas outlet, so as to dilute the ozone concentration and quickly diffuse the ozone gas to the surrounding environment to make the environmental ozone concentration uniform. The ultrasonic humidifier vibrates water using ultrasound to generate fine mist. The humidification fan blows the air through the humidification pipe to bring out the mist from the humidification outlet, thereby increasing the environmental humidity and strengthening the disinfection and sterilization effect. According to the use environment, the size of the space and whether there is a person, the present invention sets different amounts of humidification and ozone gas to be generated through the button display panel to meet various needs.

The invention adopts an ultrasonic humidifier and an electrolytic ozone generator. According to different space sizes and sterilization requirements, through the button display panel, the control board independently controls the ultrasonic humidifier and the electrolytic ozone generator to generate different amounts of humidification and ozone gas to be generated. When there is a person in an indoor space, the current of the sterilization and disinfection device is controlled to be decreased so as to decrease ozone gas to be generated for the ozone concentration to be less than 0.08 ppm, thereby meeting the standard of ozone concentration for human safety in the indoor space. When there is no person in the indoor space, the current of the sterilization and disinfection device is controlled to be increased so as to increase ozone gas to be generated for the ozone concentration to be 0.1-0.5 ppm, thereby achieving a sterilization and disinfection effect against the new novel coronavirus and other harmful bacteria and viruses in the indoor space.

### BRIEF DESCRIPTION OF THE DRAWINGS

FIG. 1 is a block diagram of the present invention;

FIG. 2 is a schematic view showing the internal structure of the present invention;

FIG. 3 is another schematic view showing the internal structure of the present invention; and

FIG. 4 is a perspective view of the present invention.

### DETAILED DESCRIPTION OF THE PREFERRED EMBODIMENTS

Embodiments of the present invention will now be described, by way of example only, with reference to the accompanying drawings.

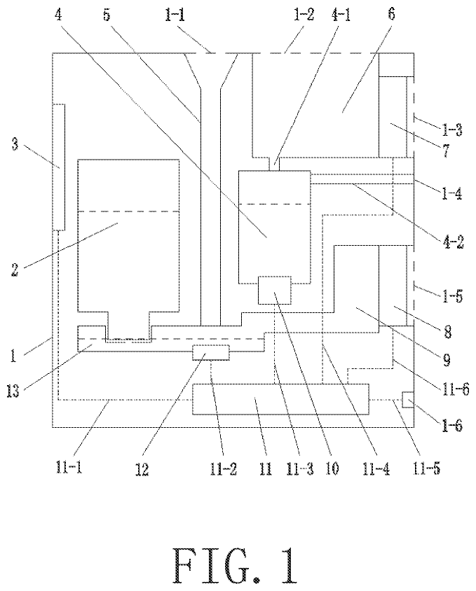

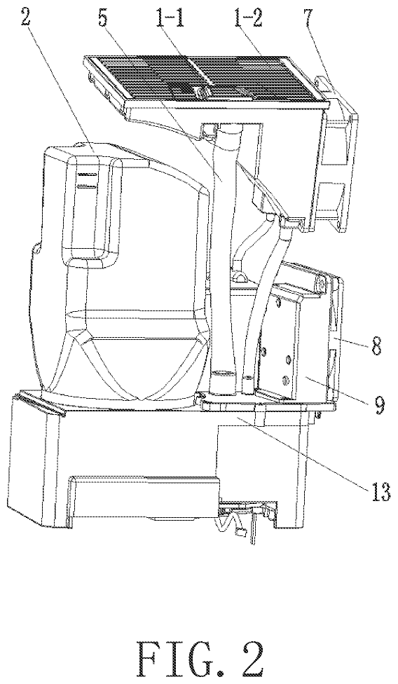

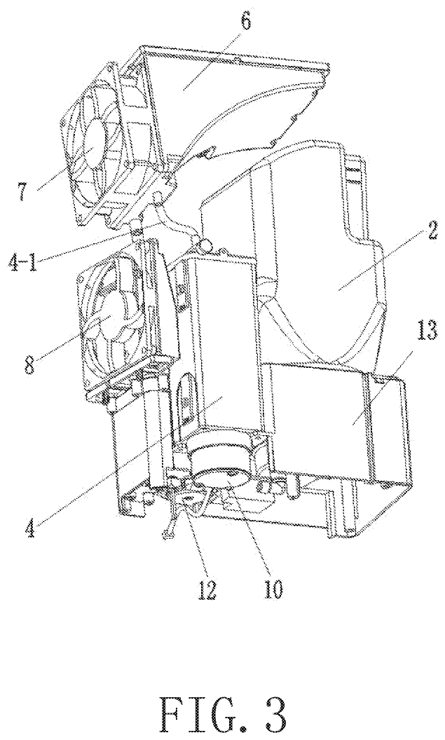

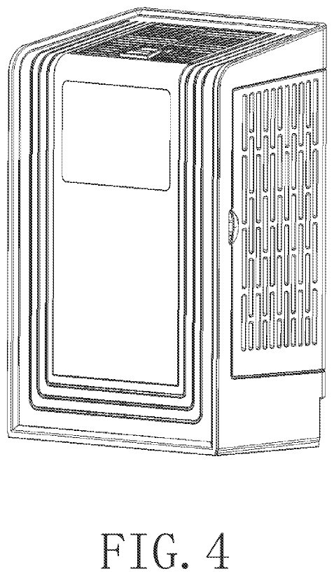

As shown in FIGS. 1-4, the present invention discloses a sterilization and disinfection device against the BACTERIA AND VIRUSES, comprising a housing 1. A humidification water tank 2 and a humidification water trough 3 are provided inside the housing 1. The humidification water tank 2 is arranged above the humidification water trough 13. The mouth of the humidification water tank 2 is inserted into the humidification water trough 13. An ultrasonic humidifier 12 is mounted to the bottom of the humidification water trough 13. One end of a humidification pipe 5 is connected to an upper outlet of the humidification water trough 13, and the other end of the humidification pipe 5 is connected to a humidification outlet 1-1 on the housing 1. One end of a humidification air input channel 9 is connected to the upper end of the humidification water trough 13, and the other end of the humidification air input channel 9 is connected to a humidification fan 8. The humidification fan 8 is installed in a humidification air inlet 1-5 on the housing 1. An ozone water tank 4 is provided inside the housing 1. An electrolytic ozone generator 10 is mounted to the bottom of the ozone water tank 4. One end of an ozone gas output pipe 4-1 is connected to the top of the ozone water tank 4, and the other end of the ozone gas output pipe 4-1 is connected to a mixing chamber 6. The top of the mixing chamber 6 communicates with an ozone gas outlet 1-2. One end of a water filling pipe 4-2 is connected to the top of the ozone water tank 4, and the other end of the water filling pipe 4-2 is connected to a water filling port 1-4 on the housing 1. An ozone fan 7 is mounted to one side of the mixing chamber 6. The ozone fan 7 is located in an ozone air inlet 1-3 on the housing 1. A power inlet 1-6 and a button display panel 3 are provided on the housing 1. A control board 11 is provided inside the housing 1. The control board 11 is electrically connected to the button display panel 3 through a first wire 11-1. The control board 11 is electrically connected to the ultrasonic humidifier 12 through a second wire 11-2. The control board 11 is electrically connected to the electrolytic ozone generator 10 through a third wire 11-3. The control board 11 is electrically connected to the ozone fan 7 through a fourth wire 11-4. The control board 11 is electrically connected to the humidification fan 8 through a sixth wire 11-6. The control board 11 is electrically connected to the power inlet 1-6 through a fifth wire 11-5.

When a space needs to be sterilized and disinfected, the control board 11 is powered through the power inlet 1-6, the humidification water tank 2 is filled with water, and the water flows into the humidification water trough 13. The ozone water tank 4 is filled with water through the water filling port 1-4 and the water filling pipe 4-2 on the housing 1. According to the space size and environmental requirements, different amounts of moisture and ozone gas to be generated can be set through the button display panel 3. The control board 11 controls the power supply time of the ultrasonic humidifier 12 through the second wire 11-2 according to the setting to control the amount of moisture. When the ultrasonic humidifier 12 is running, the humidifying fan 8 is also running at the same time. The air flows from the humidification air inlet 1-5 and the humidification air input channel 9 on the housing 1 to send water vapor to the space through the humidification pipe 5 and the humidification outlet 1-1 to increase the moisture of the space. The control board 11 controls the power supply current of the electrolytic ozone generator 10 through the third wire 11-3 according to the setting to control the amount of ozone gas to be generated. The ozone gas generated by the electrolytic ozone generator 10 flows to the mixing chamber 6 through the ozone gas output pipe 4-1. When the electrolytic ozone generator 10 is running, the ozone fan 7 is also running at the same time. The air enters the mixing chamber 6 through the ozone air inlet 1-3 on the housing 1 and is mixed with ozone gas and then sent to the space through the ozone gas outlet 1-2. When there is a person in an indoor space, the current of the sterilization and disinfection device is controlled to be decreased so as to decrease ozone gas to be generated for the ozone concentration to be less than 0.1 ppm, thereby meeting the standard of ozone concentration for human safety in the indoor space. When there is no person in the indoor space, the current of the sterilization and disinfection device is controlled to be increased so as to increase ozone gas to be generated for the ozone concentration to be 0.2-0.5 ppm, thereby achieving a sterilization and disinfection effect against the BACTERIA AND VIRUSES in the indoor space.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A sterilization and disinfection device, comprising a housing (1), housing (1) having a power inlet (1-6) and a button display panel (3) thereon, a control board (11) being provided inside the housing (1), a humidification water tank (2) being provided inside the housing (1), the humidification water tank (2) being arranged above a humidification water trough (13); the humidification water tank (2) having a mouth to be inserted into the humidification water trough (13), an ultrasonic humidifier (12) being mounted to a bottom of the humidification water trough (13), one end of a humidification pipe (5) being connected to an upper outlet of the humidification water trough (13), another end of the humidification pipe (5) being connected to a humidification outlet (1-1) on the housing (1), one end of a humidification air input channel (9) being connected to an upper end of the humidification water trough (13), another end of the humidification air input channel (9) being connected to a humidification fan (8), the humidification fan (8) being installed in a humidification air inlet (1-5) on the housing (1); an ozone water tank (4) being provided inside the housing (1), an electrolytic ozone generator (10) being mounted to a bottom of the ozone water tank (4), one end of an ozone gas output pipe (4-1) being connected to a top of the ozone water tank (4), another end of the ozone gas output pipe (4-1) being connected to a mixing chamber (6), a top of the mixing chamber (6) communicating with an ozone gas outlet (1-2), one end of a water filling pipe (4-2) being connected to the top of the ozone water tank 4, another end of the water filling pipe (4-2) being connected to a water filling port (1-4) on the housing (1), an ozone fan (7) being mounted to one side of the mixing chamber (6), the ozone fan (7) being located in an ozone air inlet (1-3) on the housing (1).

2. The sterilization and disinfection device as claimed in claim 1, wherein when there is a person in an indoor space, the current of the sterilization and disinfection device is controlled to be decreased so as to decrease ozone gas to be generated for an ozone concentration to be less than 0.08 ppm.

3. The sterilization and disinfection device as claimed in claim 1, wherein when there is no person in an indoor space, the current of the sterilization and disinfection device is controlled to be increased so as to increase ozone gas to be generated for an ozone concentration to be 0.1-0.5 ppm.

4. The sterilization and disinfection device as claimed in claim 1, wherein the control board (11) is electrically connected to the button display panel (3), the ultrasonic humidifier (12), the electrolytic ozone generator (10), the ozone fan (7), the humidification fan (8) and the power inlet (1-6), respectively.
